(19) United States
(12) Patent Application Publication
(10) Pub. No.: US 2014/0018822 A1
(43) Pub. Date: Jan. 16, 2014

(54) SURGICAL POSITIONING ASSEMBLY AND SURGICAL INSTRUMENT

(71) Applicant: David Main, Leeds (GB)

(72) Inventor: David Main, Leeds (GB)

(21) Appl. No.: 14/007,928

(22) PCT Filed: Mar. 26, 2012
(86) PCT No.: PCT/GB12/50657

§ 371 Date: Sep. 26, 2013

(30) Foreign Application Priority Data
Mar. 31, 2011 (GB) 1105430.1

(51) Int. Cl. A61B 17/29

(52) U.S. Cl. CPC A61B 17/2909
USPC 606/130

(57) ABSTRACT

A surgical positioning assembly for use with a surgical instrument which has an elongated shaft defining a longitudinal axis, for example a minimally invasive surgical instrument. The positioning assembly comprises: a base member comprising a skin contact surface, wherein the base member defines an opening for the elongated shaft; a rotating assembly configured to rotate relative to the base member about a rotation point located below the skin contact surface wherein the rotating assembly defines an opening for the elongated shaft; and a locking mechanism for locking the position of the positioning assembly on the elongated shaft and for locking the orientation of the rotating assembly relative to the base member.

The present invention relates a surgical positioning assembly for controlling the relative position and orientation of a surgical instrument relative to a patient's skin. The present invention also relates to a surgical instrument including the surgical positioning assembly.

Percutaneous surgical procedures, in which the surgical procedure is performed through the skin of a patient, are known. In such procedures access may be gained by puncturing the abdominal wall with a needle, the puncture then provides access for minimally invasive surgical procedures to be carried out.

US-2010/0292724 discusses minimally invasive surgical clamps, assemblies and methods. A surgical clamp may comprise an outer hollow needle and a coaxial grasper located within the hollow needle. The hollow needle can be used to puncture a patient's skin and abdominal wall. Once the end of the hollow needle is through the abdominal wall, the coaxial grasper may be extended and used within the abdominal cavity. The graspers include end effectors which are biased into an open position that is larger than the diameter of the hollow needle so that as they are extended they open. Retracting the graspers closes the end effectors.

To facilitate the use of the surgical clamp at various orientations, US-2010/0292724 provides a suction cup with a top proximal hole and a plurality of bayonet-type grooves through which the needle can be manoeuvred. The suction cup is positioned outside the body of the patient and engages the skin of the patient. It allows the needle to be held at different angles relative to a patient.

While the suction cup provided in US-2010/0292724 may help to position the needle relative to the patient's body, angular movement places stress on the abdominal wall. The abdominal wall is relatively thick, possibly 14 mm or thicker and so changing the orientation will pull on the abdominal wall and place it under stress. This can reduce the freedom of movement of the surgical clamp.

It would therefore be desirable to provide an improved positioning assembly for a minimally invasive surgical instrument. Accordingly the present invention provides a positioning assembly for a surgical instrument wherein the positioning assembly is configured to contact the skin of a patient and enable rotation of the surgical instrument about a rotation point which is below the skin of the patient. The applicant has found that the stress on the abdominal wall is reduced if the rotation point is below the skin of the patent. As a further benefit the reduced stress allows easier movement of the surgical instrument.

According to an aspect of the present invention, there is provided surgical positioning assembly for use with a surgical instrument having an elongated shaft which defines a longitudinal axis, wherein the positioning assembly comprises:

a base member comprising a skin contact surface, wherein the base member defines an opening for the elongated shaft;

a rotating assembly configured to rotate relative to the base member about a rotation point located below the skin contact surface, wherein the rotating assembly defines an opening for the elongated shaft; and

a locking mechanism for locking the position of the positioning assembly on the elongated shaft and for locking the orientation of the rotating assembly relative to the base member.

In use the skin contact surface is placed in contact with the patient's skin. The rotating assembly can therefore rotate about a point which is below the patient's skin. In other words, if a proximal direction is towards a user of the surgical instrument and a distal direction is away from a user of the surgical instrument, then the rotation point is located distal from the skin contact surface. In use, the insertion depth of the surgical instrument can be adjusted by moving it proximally and distally along the longitudinal axis through the openings defined by the base member and rotating assembly. The orientation of the surgical instrument can be adjusted by rotating the rotating assembly relative to the base member.

In prior art devices, such as described in US-2010/0292724, the equivalent rotation point was above the surface of the patient's skin in use, leading to increased stress on the abdominal wall in use. This also limits the freedom of movement.

The locking mechanism may be a single locking mechanism for locking both the position of the positioning assembly on the elongated shaft and the orientation of the rotating assembly relative to the base member. The single locking mechanism may lock the position and orientation substantially simultaneously.

Preferably, the rotation point is at least 2 mm below the skin contact surface. More preferably, the rotation point is at least 5 mm below the skin contact surface. The rotation point may be no more than no more than 40 mm, more preferably no more than 25 mm below the skin contact surface. In some embodiments the distance of the rotation point from the skin contact surface is chosen such that it is positioned within the abdominal wall of a patient in use. In other embodiments the distance of the rotation point from the skin contact surface may chosen so that the rotation point is below the abdominal wall in use.

The locking mechanism may comprise means for reducing the effective diameter of the opening defined by the rotating assembly. This enables the locking mechanism to lock the position of the positioning assembly along the longitudinal axis of the surgical instrument. For example, it may ensure that the surgical instrument is not inserted further into the patient.

The locking mechanism may comprise means for the rotating assembly to engage the base member. This enables the orientation of the surgical instrument relative to the positioning assembly to be locked. If the rotating assembly engages the base member a friction lock may be formed to prevent rotation of the rotating assembly relative to the base member.

The opening defined by the rotating assembly may be configured to position the rotation point on the longitudinal axis, and the locking mechanism may comprise a lever for rotation about the longitudinal axis, wherein the lever is moveable between a locked position and an unlocked position. This allows the locking mechanism to be easily locked and unlocked.

The lever may define an opening for receiving the elongated shaft which is not symmetrical about a plane containing the longitudinal axis. The non-symmetrical opening on the locking lever may combine with the openings on the rotating assembly to define an opening having a reduced effective diameter in the locked position and a larger effective diameter in other positions. This enables locking in the direction of the longitudinal axis in the locked position.

The lever may define an opening for receiving the elongated shaft which is offset from the longitudinal axis. This offset opening may combine with the openings on the rotating assembly to define an opening having a reduced effective diameter in the locked position and a larger effective diameter in other positions. This enables locking in the direction of the longitudinal axis in the locked position.

The lever may comprise a first locking surface, wherein at least a part of the locking surface is angled relative to a plane perpendicular to the longitudinal axis, and the rotating assembly may comprise a locking surface and wherein, when the lever is in the locked position, the first locking surface is engaged by the second locking surface. The engagement of the first and second locking surfaces can alter the position of the lever on the longitudinal axis, this can force the lever closer to the base member so that it engages the base member to create a friction lock preventing rotation of the rotating assembly relative to the base member. The angled surface may function as a wedge. The angled surface may be flat or curved or have any other suitable profile. For example a curved profile may provide a cam action to the locking. At least a part of the second locking surface may also be angled relative to a plane perpendicular to the longitudinal axis.

The lever may further comprise a protrusion extending towards the base member. This can amplify the friction lock with the base member by increasing the pressure exerted on the base member when the angled surfaces are engaged.

The rotating assembly comprises at least one radial extension. In use, the radial extension may contact the patient's skin to limit relative rotation and avoid placing undue stress on a patient's abdominal wall. Preferably, a plurality of radial extensions are provided and are evenly spaced around the longitudinal axis. For example, in one embodiment, four radial extensions may be provided each separated by 90 degrees.

The base member may comprise a first articulation surface which is a portion of a sphere centred on the rotation point and the rotating assembly may be configured to move relative to the articulation surface. This provides a simple construction which allows control of the position of the rotation point.

The base member may further comprise a second articulation surface below the first articulation surface such that the first and second articulation surfaces define a portion of a spherical shell. The rotating assembly may then comprise a first part to engage the first articulation surface and a second part to engage the second articulation surface. This allows the rotating assembly to be attached securely to the base member. A further advantage of this construction is that the portion of the spherical shell will define a recess that can contain the second part of the rotating assembly.

According to another aspect of the present invention, there is provided a surgical instrument comprising an elongated shaft defining a longitudinal axis; and a surgical positioning assembly as discussed above, with or without the optional features also discussed above.

The surgical instrument may be a percutaneous surgical instrument. The surgical instrument may be an insert for connection to a separate handle or comprise a handle. The surgical instrument may further comprise an operative portion at its distal end. The operative portion may be a grasper, clamp, dissector or any other minimally invasive surgical instrument.

Embodiments of the invention will now be described by way of example only, with reference to the accompanying drawings, in which:

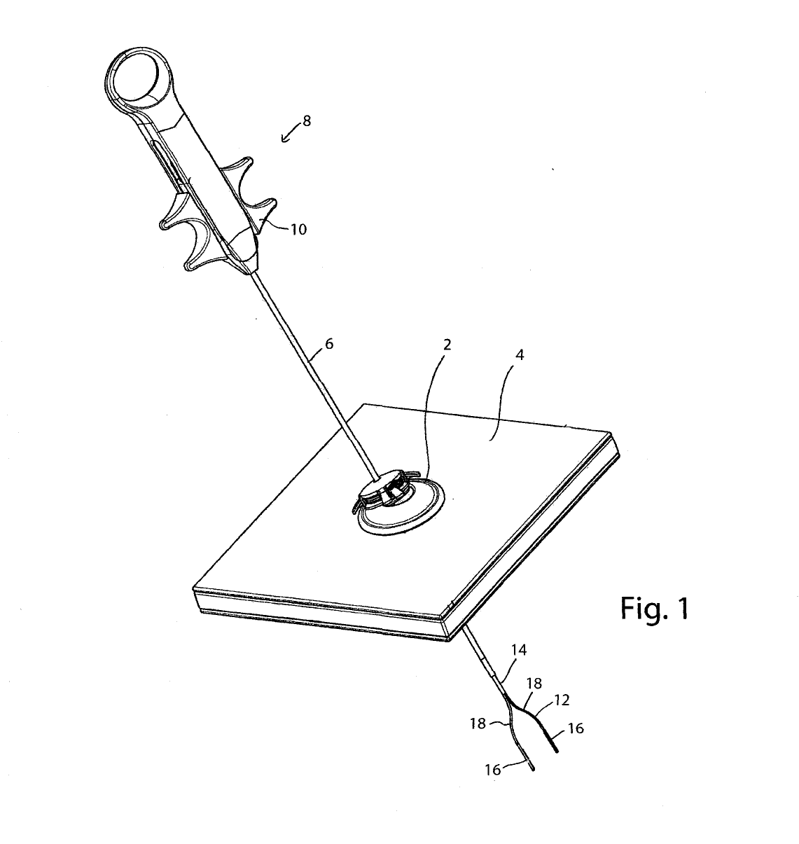

FIG. 1 is a perspective view of a surgical instrument with a positioning assembly according to a first embodiment in position through an abdominal wall;

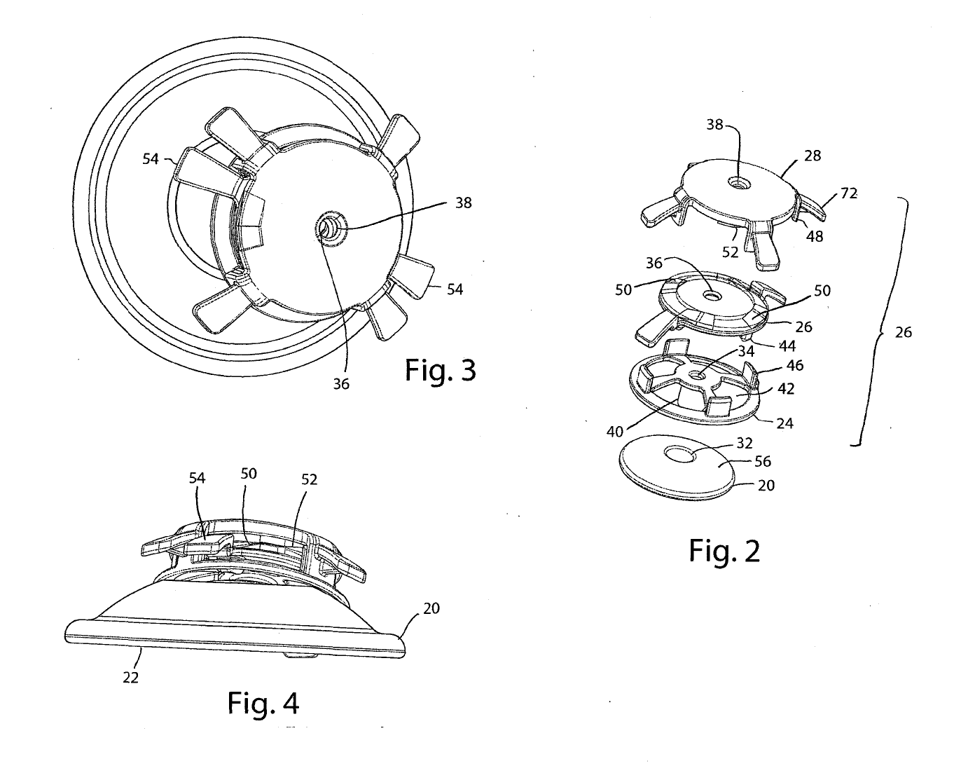

FIG. 2 depicts an exploded view of parts of the positioning assembly of FIG. 1;

FIG. 3 depicts a top perspective view of the positioning assembly of FIG. 1 in the locked position;

FIG. 4 depicts a side perspective view of the positioning assembly of FIG. 1 in the locked position;

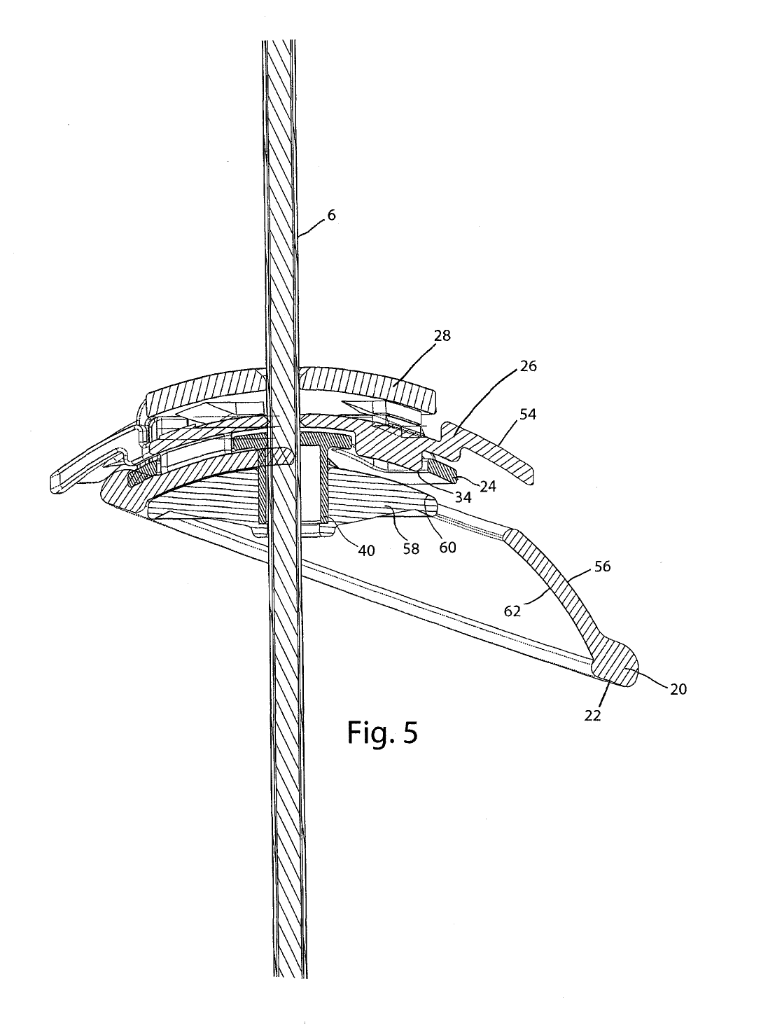

FIG. 5 is a partial cross section through the positioning assembly of FIG. 1 in place on an elongated shaft of a surgical instrument;

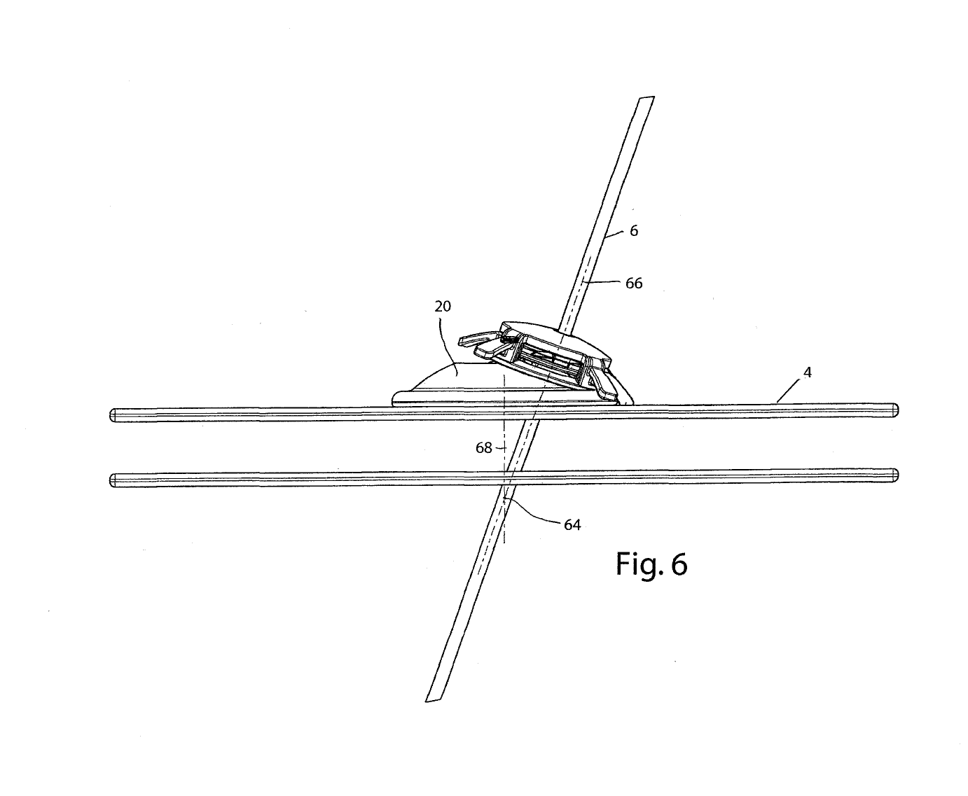

FIG. 6 is a diagrammatic representation depicting the position of the rotation point in the embodiment in FIG. 1;

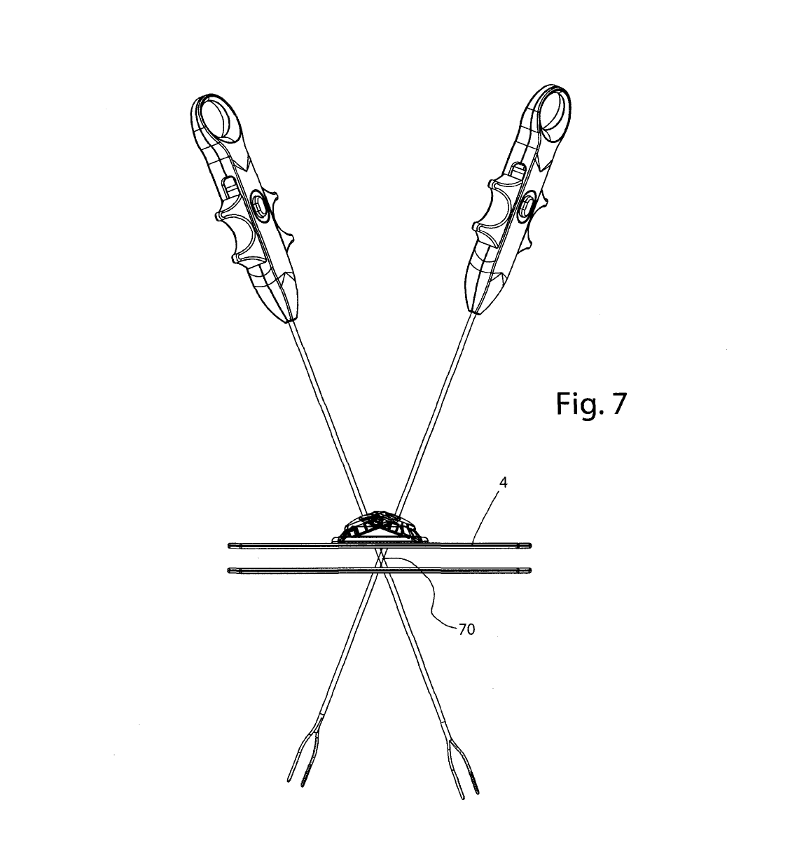

FIG. 7 is a diagrammatic representation depicting the position of the rotation point in a another embodiment of the invention.

FIG. 1 depicts a perspective view of a surgical instrument with a positioning assembly 2 according to an embodiment of the present invention, shown extending through a diagrammatic representation of an abdominal wall 4. The positioning assembly 2 is located on an elongated shaft 6 of the surgical instrument. The elongated shaft 6 extends from a handle 8 which comprises a slider 10. The slider 10 comprises two sets of protrusions for engaging one or more of a user's digits, for example engaging a user's fingers in use.

The elongated shaft 6 is a hollow tube within which an operative portion 12 of a surgical instrument is contained. The operative portion 12 extends from an inner rod 14 which is located within the hollow tube of the elongate shaft 6. A proximal end of the inner rod 14 is connected to the slider 10, allowing the operative portion 12 to be advanced from and retracted into the hollow tube 6.

The surgical instrument 12 is formed from a highly elastic material, for example a super elastic material, such as a super elastic alloy of nickel and titanium and is biased into an open configuration when unconstrained by the elongate shaft 6. The open configuration is depicted in FIG. 1. When the slider 10 is moved proximally, to retract the inner rod 14 and operative portion 12 into the elongate shaft, the elongate shaft 6 constrains and closes the operative portion 12.

In this embodiment, the operative portion 12 is a grasper, for grasping items within the abdominal cavity. As can be seen in FIG. 1, the operative portion 12 comprises distal, parallel portions 16. A transition portion 18 extends from the inner member 14 and defines the open position of the operative portion 12. Therefore, when the inner member 14 is withdrawn by operating the slider 10, the parallel portions 16 will be forced together while still extending out of the distal end of the elongated shaft 6. This enables them to be used to grasp items within the abdominal cavity.

Moving on to FIGS. 2, 3, 4 and 5, the construction and operation of the positioning assembly 2 will be described in more detail. FIG. 2 depicts an exploded view of some components of the positioning assembly. FIG. 3 depicts an assembled top perspective view of the components depicted in FIG. 2. FIG. 4 depicts an assembled side perspective view of the components depicted in FIG. 2.

The positioning assembly comprises a base member 20 with a lower skin contacting surface 22 for contacting a patient's skin in use. On top of the base member, a first component 24 of a rotating assembly 26 is provided. On top of the first component 24 a rotatable locking member 26 is sandwiched between a second component 28 of the rotating assembly. All of the base member 20, first component 24, locking member 26 and second component 28 define openings 32, 34, 36, 38 for receiving the elongated shaft 6. The first component 24 includes a downwardly facing projection 40, which is for engaging a second part of the rotating assembly located under the base member 20 (not shown in FIGS. 2-4, but described below with reference to FIG. 5).

The first component 24 also defines openings 42 for receiving a projection 44 formed on the locking member 26. Finally, the first component 24 also comprises upwardly facing projections 46 for engaging corresponding downward facing projections 48 provided on the second component 28. When assembled, upwardly facing projections 46 snap-fit with downwardly facing projections 48 so that the locking member 26 is contained between first component 24 and second component 28. Together, first component 24 and second component 28 define a first part of a rotating assembly.

The locking member 26 comprises an opening 36 which is offset from the axis of the openings 34, 38 in the first component 24 and second component 28. This means that as the locking member 26 is rotated, the position of opening 36 changes relative to the position of openings 34, 38. Thus, the effective diameter of the opening through the entire first part of the rotating assembly is adjusted by the rotational position of locking member 26.

Locking member 26 also comprises a locking surface 50 for engaging a corresponding locking surface 52 on the second component 28 depending on the rotational position of the locking member 26. Both locking surface 50 and locking surface 52 comprise a portion which is angled relative to a plane perpendicular to the longitudinal axis of the elongated shaft 6.

Also depicted in FIG. 2 are radial extensions 72, extending radially from the second component 28 of the rotating assembly. Radial extensions 72 have dimensions such that they may contact the skin of the patient to limit the maximum angle of the elongated shaft 6 relative to the skin contacting surface 22. This provides a guard against the instrument being orientated at too large an angle, which could overstress the abdominal wall and cause injury to the patient.

Referring to FIG. 3, the way in which the rotating assembly 26 is locked relative to a particular position on a longitudinal axis of an elongated shaft 6 will be explained. FIG. 3 depicts a perspective view of the assembled components of FIG. 2. The locking member 26 defines lever portions 54 which extend radially outwards. These are used to access and rotate locking member 26. As depicted in FIG. 3, rotating member 26 is in a locking position. It can be seen that the opening 36 of the rotating member 26 is not aligned with the opening 38 of the second component. This narrows the effective diameter of the opening and causes the rotating assembly to lock onto the elongated shaft 6.

Referring to FIG. 4, which is a side perspective view of the assembled components of FIG. 2, the way in which the orientation of the rotating mechanism is locked will now be described. As can be seen most clearly in FIG. 2, the base member 20 defines a first articulation surface 56 which defines a portion of a sphere. The underside of first component 24 of the rotating assembly has a curvature matching the curvature of the articulation surface 56. This enables the rotating assembly to be translated over the articulating surface, resulting in changing the orientation of the surgical instrument relative to the base member 20 and therefore the patient's skin. In order to lock the rotating assembly relative to the base member 20, the locking member 26 comprises locking surfaces 50, a portion of which is angled relative to the plane of the longitudinal axis, and downwardly facing projections 44. As the locking member 26 is rotated by lever 54 into the locking position (shown in FIG. 4), locking surface 50 engages locking surface 52 and together they provide a wedge action to push locking member 50 downwardly, away from the second component 28, in the direction of the longitudinal axis. This has the effect of pushing projection 44 downwards, where it engages the articulation surface 56, forming a friction lock and locking the orientation of the rotating assembly relative to the base member 20.

Referring now to FIG. 5, which is a partial cross-section, the way in which the rotating assembly is attached to the base member 20 can be understood more clearly. The downward projection 40 of the first component 24 is connected to a second part 58 of the rotating assembly. Between them, the lower surface of the first component 24 and the upper surface 60 of the second part 58 sandwich a spherical shell portion of the base member 20 defined by upper articulation surface 56 and a lower articulation surface 62. When locking member 26 is in the unlocked position, the rotating assembly is positioned on the base member securely, so that it cannot be removed, but so that it can translate over the hemispherical surface 56.

When the locking member 26 is moved to the locking position, the interaction of locking surfaces 50, 52 push the locking member 26 along the longitudinal axis, away from the user. This pushes projections 44 down, through openings 42 in the first component 24 to engage the articulation surface 56 of the base member 20. In this position, the second portion 58 does not move, so the articulation shell is held tightly between the projections 44 and the second portion 58. This creates a friction lock, locking orientation.

The locking member 26 is configured so that, in the locking position, the off-centre opening 36 locks longitudinal position on the shaft at the same time as the rotational position is locked by locking surface 50. Therefore, by rotation of the locking member 26 the positioning assembly can be locked both in its position along the longitudinal axis of the elongated shaft 6 and also in its orientation relative to the base member.

In the present invention, the positioning mechanism is configured so that the rotating assembly rotates about a point which is located below the skin contact surface 22. FIG. 6 depicts the location of the rotation point 64 in the embodiment of FIG. 1. It can be seen that the rotation point is positioned sufficiently far from the skin contact surface 22 that it is positioned below the abdominal wall 4 of the patient in use. In this embodiment, the centre of curvature of the spherical shell portion of the base member 20 defines the rotation point: it is the centre of the sphere described by the curved surface 56 if that surface defined an entire sphere, rather than only a portion of the sphere. Positioning the rotation point 64 below the abdominal wall minimises the stress applied to the internal side of the abdominal wall and improves manoeuvrability of the instrument with less discomfort to a patient.

FIG. 6 also illustrates how the longitudinal axis 66 of the instrument shaft 6 intercepts the rotation point 64 together with the central axis 68 of the base member 20.

FIG. 7 depicts a diagrammatic representation of an alternative embodiment of the invention, where the rotation point is located within the abdominal wall. The construction of the embodiment of FIG. 7 is the same as described above for the embodiment of FIG. 1. However, in this embodiment, the rotation point 70 is positioned within the abdominal wall 4, generally at a mid point of the abdominal wall. This can minimise stress on the abdominal wall, because both an upper and lower surface of the abdominal wall will be equally stressed when the orientation of the shaft is changed. This may allow more freedom to operate and reduce patient's discomfort.

A surgical instrument has been described which provides an improved positioning means for allowing selective locking of the position and orientation of an instrument relative to a patient's skin. It will be appreciated that although the above embodiments describe a surgical instrument which provided a grasper, it can equally be applied to other forms of surgical instruments using an operative portion, for example, dissectors. It will also be appreciated that this principle could be applied to any minimally invasive surgical instrument, for example surgical instruments comprising an operative portion which is actuated by moving an inner rod relative to the outer shaft, where the operative portion comprises a first part which is connected to the outer shaft and a second part which is connected to the inner rod, such that relative movement of the inner rod to the outer shaft actuates the operative portion.

1. A surgical positioning assembly for use with a surgical instrument having an elongated shaft which defines a longitudinal axis, wherein the positioning assembly comprises:
a base member comprising a skin contact surface, wherein the base member defines an opening for the elongated shaft;
a rotating assembly configured to rotate relative to the base member about a rotation point located below the skin contact surface wherein the rotating assembly defines an opening for the elongated shaft; and
a locking mechanism for locking the position of the positioning assembly on the elongated shaft and for locking the orientation of the rotating assembly relative to the base member.

2. A surgical positioning assembly according to claim 1, wherein the rotation point is at least 2 mm below the skin contact surface.

3. A surgical positioning assembly according to claim 1, wherein the locking mechanism comprises means for reducing the effective diameter of the opening defined by the rotating assembly.

4. A surgical positioning assembly according to claim 1, wherein the locking mechanism comprises means for the rotating assembly to engage the base member.

5. A surgical positioning assembly according to claim 1, wherein the opening defined by the rotating assembly is configured to position the rotation point on the longitudinal axis, and the locking mechanism comprises a lever for rotation about the longitudinal axis, wherein the lever is moveable between a locked position and an unlocked position.

6. A surgical positioning assembly according to claim 5, wherein the lever defines an opening for receiving the elongated shaft which is not symmetrical about a plane containing the longitudinal axis.

7. A surgical positioning assembly according to claim 5, wherein the lever defines an opening for receiving the elongated shaft which is offset from the longitudinal axis.

8. A surgical positioning assembly according to claim 5, wherein:
the lever comprises a first locking surface, wherein at least a portion of the first locking surface is angled relative to a plane perpendicular to the longitudinal axis;
the rotating assembly comprises a second locking surface; and
when the lever is in the locked position, the first locking surface is engaged by the second locking surface.

9. A surgical positioning assembly according to claim 8, wherein the lever further comprises a protrusion extending towards the base member.

10. A surgical positioning assembly according to claim 1, wherein the rotating assembly comprises at least one radial extension.

11. A surgical positioning assembly according to claim 1, wherein the base member comprises a first articulation surface which is a portion of a sphere centred on the rotation point and wherein the rotating assembly is configured to move relative to the articulation surface.

12. A surgical positioning assembly according to claim 11, wherein the base member further comprises a second articulation surface below the first articulation surface such that the first and second articulation surfaces define a portion of a spherical shell and wherein the rotating assembly comprises a first part to engage the first articulation surface and a second part to engage the second articulation surface.

13. A surgical instrument comprising:
an elongated shaft defining a longitudinal axis; and
a surgical positioning assembly according to claim 1.
